(19) United States
(12) Patent Application Publication
(10) Pub. No.: US 2017/0319532 A1
(43) Pub. Date: Nov. 9, 2017

(54) METHODS AND FORMULATIONS FOR TREATMENT AND/OR PREVENTION OF BLOOD-ASSOCIATED DISORDERS

(71) Applicant: TWi Biotechnology, Inc., Taipei (TW)

(72) Inventors: Chih-Kuang Chen, Taipei (TW); Jing-Yi Lee, Taipei (TW); Wei-Shu Lu, Taipei (TW); Carl Oscar Brown, III, San Diego, CA (US)

(21) Appl. No.: 15/587,510

(22) Filed: May 5, 2017

Related U.S. Application Data
(60) Provisional application No. 62/332,776, filed on May 6, 2016.

(51) Int. Cl. A61K 31/222; A61K 45/06

(52) U.S. Cl. CPC A61K 31/222; A61K 45/06

(57) ABSTRACT

A method of treating and/or preventing blood-associated disorders is provided. Also provided is a method of treating and/or preventing hemophilic arthropathy and/or hemochromatosis arthropathy in a subject.

### BACKGROUND OF THE INVENTION

### Field of the Invention

The present invention relates to methods of treating and/or preventing blood-associated disorders, especially to a method of treating and/or preventing hemophilic arthropathy or hemochromatosis arthropathy. The invention also provides pharmaceutical formulations for use in the methods of the invention.

### Descriptions of the Related Art

Hemophilia is an X-linked congenital bleeding disorder caused by a deficiency of coagulation factor VIII in hemophilia A or factor IX in hemophilia B. The worldwide prevalence of hemophilia A is 1 in 5,000 males and for hemophilia B is 1 in 30,000 males. The characteristic phenotype in hemophilia is the bleeding tendency. The severity of bleeding in hemophilia generally correlates inversely with the clotting factor level. Most bleeding occurs internally, into the joints or muscles.

Hemophilic arthropathy (HA) is a disabling and common complication of severe (and, to a lesser extent, moderate) hemophilia, in which a characteristic chronic arthropathy develops as a result of recurrent bleeding into joints, particularly knee, ankle, and elbow joints. HA is the primary cause of morbidity in the hemophilic population. The rate of progression of this disease is determined mainly by the number of hemarthroses, or joint bleeding events.

Cumulative evidence indicates that the pathogenesis of HA is probably multifactorial, and may include degenerative cartilage-mediated and inflammatory synovium-mediated components. Recurrent hemorrhage into the joint leads to deposition of hemosiderin, an iron complex that forms following phagocytosis of red blood cells, which may have a direct degenerative toxic effect on the cartilage. Further, synovial inflammation and subsequent hypertrophy increases the risk of bleeding events, contributing to a vicious cycle. Repeated bleeding and chronic synovitis lead to the progressive destruction of the cartilage and subchondral bone. These abnormalities include loss of the joint space, subchondral bone irregularity, joint surface erosions, and subchondral cyst formation. These changes ultimately result in severe functional impairment.

Optimal management of hemophilic joint disease requires early prevention and treatment of acute joint bleeds before the onset of degenerative disease. Early treatment of joint hemorrhages can be achieved with replacement clotting factor concentrates. Moreover, early prophylaxis with factor concentrates in children has been shown to prevent not only joint bleeding but also improve joint outcomes, particularly in those with severe hemophilia. However, despite the success of factor replacement therapy, intra-articular bleeding is still a major clinical problem of the disease, particularly in those with severe hemophilia or clotting factor inhibitors.

Adjunctive management includes analgesics for pain relief, anti-inflammatory drugs for synovitis, physiotherapy to help preserve movement and function of the joints, and surgical treatment for patients with severe joint impairment where conservative therapies have failed.

When the acute phase of the joint bleeding is over, treatment of the synovitis that often develops must be considered. The role of chronic use of systemic non-steroidal anti-inflammatory drugs (NSAIDs) or corticosteroids for chronic synovitis in HA is limited due to their side-effects and lack of confirmatory efficacy evidence. NSAIDs or corticosteroids can relieve pain only, but cannot significantly reduce joint destruction. So far, there is no effective therapy that can prevent or abort the development of the soft tissue and osteochondral changes in HA.

Hemochromatosis arthropathy has many clinical and joint structural features in common with HA. Both of these disorders are due to iron deposition in the affected joints, with its subsequent inflammatory and degenerative effects. Likewise, no effective therapy is currently available to prevent or abort the development of the soft tissue and osteochondral changes in hemochromatosis arthropathy.

Therefore, there is a need for specific methods and compounds that can treat and/or prevent blood-associated disorders such as HA, and hemochromatosis arthropathy.

### SUMMARY OF THE INVENTION

The primary objective of this invention is to provide a method of treating and/or preventing blood-associated disorders.

Another objective of this invention is to provide a method of treating and/or preventing hemophilic arthropathy.

Still another objective of this invention is to provide a method of treating and/or preventing hemochromatosis arthropathy.

The invention, therefore, provides methods of treating and/or preventing blood-associated disorders comprising administering to a subject in need thereof a therapeutically effective amount of a compound selected from the group consisting of diacerein, rhein, monoacetylrhein, and pharmaceutically acceptable salts or esters or prodrugs thereof.

The invention al so provides pharmaceutical formulations comprising diacerein, rhein, monoacetylrhein, and pharmaceutically acceptable salts or esters or prodrugs thereof for use in treatment and/or prevention of blood-associated disorders.

The blood-associated disorders include, but are not limited to, hemophilia A, hemophilia B, hemochromatosis arthropathy, von Willebrand disease (vWD), factor I deficiency, factor II deficiency, factor V deficiency, factor VII deficiency, factor X deficiency, factor XI deficiency, factor XIII deficiency, vitamin K deficiency, immune thrombocytopenic purpura, thrombocytopenia, disseminated intravascular coagulation, Glanzmann thrombasthenia, Bernard-Soulier syndrome, platelet granule disorders, alpha2-antiplasmin deficiency, plasminogen activator inhibitor-1 deficiency, hemorrhagic telangiectasias, drug-induced bleeding disorders, trauma-induced hemarthrosis, pigmented villonodular synovitis, Charcot arthropathy, Ehlers-Danlos syndrome, leukemia, myeloproliferative disorders, thrombocythemia, chondrosarcoma, synovial hemangioma, and synovioma.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a line graph showing that diacerein reduced joint swelling in rats with induced hemophilic arthropathy.

### DETAILED DESCRIPTION OF THE INVENTION

The term “therapeutically effective amount,” as used herein, refers to an amount that alleviates or reduces one or more symptoms of a disease.

The term “diacerein or its analogs,” as used herein, refers to diacerein, rhein, monoacetylrhein, or a salt or ester or a prodrug thereof.

The term “prodrug,” as used herein, refers to any compound that can be converted into rhein and exerts its physiological function in the form of rhein within the body.

Unless otherwise stated herein, the terms “a (an)”, “the” or the like used in this specification (especially in the Claims hereinafter) shall be understood to encompass both the singular form and the plural form.

Chemically, rhein is 9,10-dihydro-4,5-dihydroxy-9,10-dioxo-2-anthracene carboxylic acid having a structure of Formula (I), and one of its prodrugs, diacerein, is 4,5-bis (acetyloxy) 9,10-dihydro-4,5-dihydroxy-9,10-dioxo-2-anthracenecarboxylic acid having a structure of Formula (II). Diacerein is entirely converted into rhein before reaching the systemic circulation, and exerts its physiological function in form of rhein within the body.

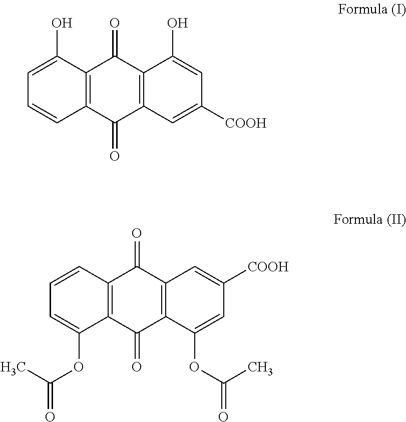 [SMILES: O=C(O)c1cc(O)c2c(c1)C(=O)c1cccc(O)c1C2=O ; CC(=O)Oc1cccc2c1C(=O)c1c(OC(C)=O)cc(C(=O)O)cc1C2=O]

Diacerein is an anti-inflammatory agent widely used in the treatment of osteoarthritis. Presently, diacerein capsules are available in 50 mg strength and are marketed under various trade names in different countries, including Art 50®, Artrodar®, etc.

The inventors of the present application found that diacerein may reduce joint swelling caused by bleeding, and thus the invention provides a method for the treatment and/or prevention of blood-associated disorders in a subject, comprising administering to the subject in need of such treatment and/or prevention a therapeutically effective amount of a compound selected from the group consisting of diacerein, rhein, monoacetylrhein, and pharmaceutically acceptable salts or esters or prodrugs thereof.

In one embodiment, the compound is administered at a dose of about 5 to 500 mg per day, and preferably about 20 to 200 mg per day. In another embodiment, the compound is administered at a dose of about 25 to 100 mg twice daily, and preferably about 50 to 75 mg twice daily.

The subject in the present method includes humans and animals. In one embodiment, the subject is a hemochromatosis patient or a hemophilia patient with, for instance, hemophilia A or hemophilia B.

In another embodiment, the subject is a patient with hemochromatosis arthropathy, von Willebrand disease (vWD), factor I deficiency, factor II deficiency, factor V deficiency, factor VII deficiency, factor X deficiency, factor XI deficiency, factor XIII deficiency, vitamin K deficiency, immune thrombocytopenic purpura, thrombocytopenia, disseminated intravascular coagulation, Glanzmann thrombasthenia, Bernard-Soulier syndrome, platelet granule disorders, alpha2-antiplasmin deficiency, plasminogen activator inhibitor-1 deficiency, hemorrhagic telangiectasias, drug-induced bleeding disorders, trauma-induced hemarthrosis, pigmented villonodular synovitis, Charcot arthropathy, Ehlers-Danlos syndrome, leukemia, myeloproliferative disorders, thrombocythemia, chondrosarcoma, synovial hemangioma, or synovioma.

In one embodiment, the method reduces or ameliorates tingling or tightness in the joint, joint pain, difficult joint movement, joint effusion, joint swelling, joint fusion, erosion of joint cartilage, subchondral changes, subchondral cyst formation, synovial hyperplasia, synovial inflammation, joint fibrosis, ankylosis, hemarthrosis, paresthesia, joint destruction, and hemosiderosis in the subject.

In one embodiment, the invention provides a method of treatment of a blood-associated disorder comprising administering to a patient in need thereof a therapeutically effective amount of a compound selected from the group consisting of diacerein, rhein, monoacetylrhein, and pharmaceutically acceptable salts or esters or prodrugs thereof, wherein said patient was previously treated with another drug for the blood-associated disorder.

When administered to a subject in need thereof, diacerein or its analogs can be prepared as a pharmaceutical composition. Pharmaceutical compositions contemplated for use for the purposes of the present invention can be in the form of a solid, solution, emulsion, dispersion, micelle, liposome and the like. The compositions may be administered using any means known in the art, such as intravenously, topically, intradermally, intramuscularly, transdermally, subcutaneously, intranasally, parenterally, intrathecally, vaginally, rectally, colorectally, orally, intracranially, retroorbitally, or intrasternally. Preferably, the compositions are adapted for oral administration. For example, the drug can be mixed with suitable excipients for the preparation of tablets, capsules, pellets, troches, lozenges, solutions, powders or granules, suspensions, hard or soft capsules and any other forms suitable for use.

In some embodiments, the subject is co-administered with one or more additional therapeutic agents suitable for the treatment of blood-associated disorders selected from the group consisting of coagulation factor VIIa, coagulation factor VIII, coagulation factor IX, acetaminophen, steroids, hyaluronic acid, glucosamine, chondroitin, shea nut oil extract (shea butter), desmopressin, anti-hemophilic factor recombinant, anti-inhibitor coagulant complex, antifibrinolytic agents, rituximab, chelation therapy, and nonsteroidal anti-inflammatory drugs (NSAIDs) including COX-2 inhibitors.

Examples of NSAIDs include, but are not limited to, 2-arylpropionic acids such as ibuprofen, ketorolac and naproxen; n-arylanthranilic acids such as mefenamic acid and meclofenamic acid; oxicams such as piroxicam and meloxicam; and arylalkanoic acids such as diclofenac, etodolac, indomethacin, and sulindac. Examples of COX-2 inhibitors include, but are not limited to, celecoxib, etoricoxib, rofecoxib, and valdecoxib. Examples of coagulation factor VIII and factor IX include, but are not limited to, Helixate, Monoclate-P, Beriate, BeneFix, Alprolix, Idelvion, corticosteroids, and Rixubis.

In some embodiments, diacerein or its analogue can be the only active agent in the compositions of the invention. The compositions of the invention may contain pharmaceutical excipients (i.e., inactive compounds) commonly used in the art.

Suitable excipients include antioxidants, gelling agents, pH adjusting agents/buffers, penetration enhancers, preservatives, chelating agents, humectants, surfactants, emulsifiers, thickeners, solvents and stabilizers. Herein, excipients/ingredients in the present invention may have multiple functions, e.g., one excipient can be used as surfactant and/or stabilizer and/or emulsifier, etc.

Examples of antioxidants include, but not limited to, one or more of vitamin C, vitamin A and alpha-lipoic acid, ascorbyl palmitate, sodium pyrosulfite, butyl hydroxy anisole (BHA), butyl hydroxy toluene (BHT), and the like.

Suitable gelling agents may include, but not limited to, one or more of guar, xanthan, and carregeenan gums, anionic, nonionic, cationic and lipophilically modified guar gums, polyacrylic acids, polymethacrylic acids, cellulose resins, polyethylene glycols, hydroxy alkyl celluloses, carboxy alkyl celluloses, polyalkylene amines, and the like.

Examples of pH adjusting agents/buffers include, but not limited to, one or more of sodium bicarbonate, potassium bicarbonate, magnesium hydroxide, magnesium lactate, magnesium gluconate, aluminum hydroxide, aluminum hydroxide/sodium bicarbonate co precipitate, amino acids, aluminum glycinate, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, sodium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and the like.

Examples of penetration enhancers includes, but not limited to, one or more of diethylene glycol monoethyl ether, dimethyl sulfoxide, propylene glycol, isopropyl myristate (IPM), cal- cipotriene, detergents, emollients, ethoxy diglycol, triacetin, propylene glycol, benzyl alcohol, sodium laureth sulfate, dimethyl isosorbide, isopropyl myristate, medium chain triglyceride oil (MCT oil), menthol, isopropyl palmitate, isopropyl isostearate, propylene glycol monostearate, lecithin, diisopropyl adipate, diethyl sebacate, oleic acid, ethyl oleate, urea, glyceryl oleate, caprylic/capric triglyceride, propylene glycol dicaprylate/dicaprate, Laureth 4, Oleth-2, Oleth-20, propylene carbonate, nonoxynol-9, 2-n-nonyl-1,3-dioxolane, C7 to C14-hydrocarbyl substituted 1,3-dioxolane, 1,3-dioxane, or acetal, and nonoxynol-15, and the like.

Preservatives can be, for instance, one or more of sodium benzoate, butylated hydroxy toluene, butylated hydroxyanisole, ethylenediamine tetraacetic acid, paraoxybenzoic acid esters, chlorobutanol, benzyl alcohol, phenylethyl alcohol, dehydroacetic acid, sorbic acid, benzalkonium chloride, benzethonium chloride, phenol, phenylmercuric nitrate, thimerosal, methyl-, ethyl-, and/or propyl-paraben.

Examples of suitable solvents include, but not limited to, one or more of alcohol, castor oil, diisopropyl adipate, ethoxylated alcohol, ethyl alcohol, fatty alcohol citrate, glycerin, 1,2,6-hexanetriol, hexylene glycol, isopropyl alcohol, isopropyl myristate, isopropyl palmitate, mineral oil, phosphoric acid, polyethylene glycol 300, polyethylene glycol 400, polyethylene glycol 1450, polyethylene glycol 8000, polyethylene glycol 1000 monocetyl ether, polyethylene glycol monostearate, polyethylene glycol 400 monostearate, polyethylene glycols, polyoxyl 20 cetostearyl ether, polyoxypropylene 15-stearyl ether, polysorbate 20, polysorbate 40, polysorbate 60, polysorbate 80, polysorbates, propylene carbonate, propylene glycol, purified water, SD alcohol 40, triglycerides of saturated fatty acids, and the like.

Suitable stabilizers or surfactants can be, for example, one or more of ionic polysorbate surfactant, Tween 20, Tween 40, Tween 60, Tween 80, nonylphenol polyethylene glycol ethers, (alkylphenol-hydroxypolyoxyethylene), Poly(oxy-1,2-ethanediyl), alpha-(4-nonylphenol)-omega-hydroxy-, branched (i.e., Tergitol® NP-40 Surfactant), nonylphenol polyethylene glycol ether mixtures (i.e., Tergitol® NP-70 (70% AQ) Surfactant), phenoxypolyethoxyethanols and polymers thereof such as Triton®, Poloxamer®, Spans®, Tyloxapol®, different grades of Brij, sodium dodecyl sulfate, cetyl alcohol, stearic acid, polyoxyl stearate, and the like.

Even if an ingredient of the provided compositions may be an active agent in prior art formulations for purposes other than treatment of blood-associated disorders, it is still considered a pharmaceutical excipient for the purposes of the provided compositions as long as this ingredient is not present at an amount sufficient to effectively treat a blood-associated disorder.

Diacerein or its analogs and the additional therapeutic agents may be contained in a single formulation or may be co-administered as separate formulations.

In another embodiment, the invention provides a method for the treatment and/or prevention of hemophilic arthropathy in a subject, comprising administering to the subject in need of such treatment and/or prevention a therapeutically effective amount of diacerein or its analogs.

In yet another embodiment, the invention provides a method for the treatment and/or prevention of hemochromatosis arthropathy in a subject, comprising administering to the subject in need of such treatment and/or prevention a therapeutically effective amount of diacerein or its analogs.

The invention also provides pharmaceutical compositions for treating and/or preventing blood-associated disorders, hemophilic arthropathy, and/or hemochromatosis arthropathy, comprising a therapeutically effective amount of diacerein or its analogs.

Hereinafter, the present invention will be further illustrated with reference to the following examples. However, these examples are only provided for illustrative purposes, but not to limit the scope of the present invention.

### EXAMPLE 1

### Animal

A total of 8 female Lewis rats were used for hemophilia arthropathy (HA) study. The animals were specific pathogen free and approximately 6 to 7 weeks old upon start dosing.

### Procedure

Animals were randomized into 2 groups on Day 1 and started to receive diacerein or vehicle treatments throughout the study of 10 days. On Day 4 and Day 8, treatment was performed 1 hour before HA model induction. For HA induction, under anesthesia with 1.5-5% isoflurane (inhalation anesthesia machine, Matrix vip 3000 isoflurane), blood was collected from orbital sinus of each rat and immediately, 0.1 ml of whole blood was intraarticular injected into the cavity of left knee using syringe with 27G needle. The blood was anti-coagulated with EDTA-2K. Body weight and joint swelling measurement were assessed 4 hours post-dosing every day.

### Joint Swelling Measurement

The longitudinal and transverse axes of both knee joints were measured with calipers on Day 1 (pre-dose), Day 4 (pre-dose), and Day 4-10 (4 hours post-dose). The oval area of knee joints and % change of joint swelling was calculated using the following formula:

Oval area=longitudinal axis×transverse axis×3.14×0.25% change of joint swelling=(Oval area left−Oval area right)/Oval area right×100%

### Results

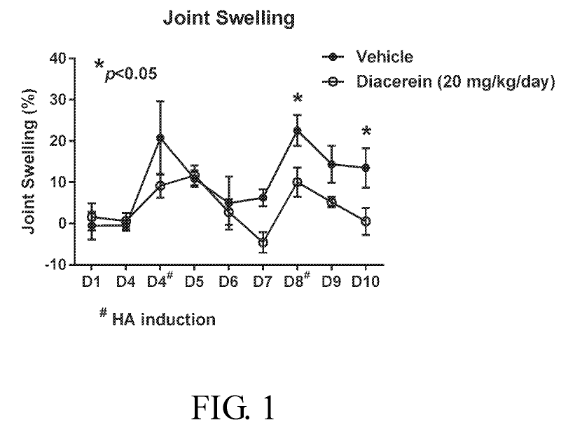

Repeated intraarticular injection of whole blood into knee joint cavity was used to induce animal hemophilia arthropathy model for this study. The result of joint swelling assessments is shown in FIG. 1. In the comparison with vehicle treatment, diacerein significantly alleviated joint swelling at Day 8, the second hemophilia arthropathy induction, of 22.57±3.77% vs. 10.02±3.54% (p<0.05) and Day 10, end of the study, of 13.51±4.78% vs. 0.50±3.28% (p<0.05).

The study results depicted that diacerein suppressed joint swelling in the hemophilic arthropathy rat model.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

1. A method for the treatment and/or prevention of blood-associated disorders in subjects, comprising administering to the subjects in need of such treatment and/or prevention a therapeutically effective amount of a compound selected from the group consisting of diacerein, rhein, monoacetylrhein, and pharmaceutically acceptable salts or esters or prodrugs thereof.

2. The method of claim 1 wherein the compound is administered at a dose of about 5 to 500 mg per day.

3. The method of claim 1 wherein the compound is administered at a dose of about 20 to 200 mg per day.

4. The method of claim 1 wherein the compound is administered at a dose of about 25 to 100 mg twice daily.

5. The method of claim 1 wherein the subject is a patient with hemophilia A, hemophilia B, von Willebrand disease (vWD), factor I deficiency, factor II deficiency, factor V deficiency, factor VII deficiency, factor X deficiency, factor XI deficiency, factor XIII deficiency, vitamin K deficiency, immune thrombocytopenic purpura, thrombocytopenia, disseminated intravascular coagulation, Glanzmann thrombasthenia, Bernard-Soulier syndrome, platelet granule disorders, alpha2-antiplasmin deficiency, plasminogen activator inhibitor-1 deficiency, hemorrhagic telangiectasias, drug-induced bleeding disorders, trauma-induced hemarthrosis, pigmented villonodular synovitis, Charcot arthropathy, Ehlers-Danlos syndrome, leukemia, myeloproliferative disorders, thrombocythemia, chondrosarcoma, synovial hemangioma, or synovioma.

6. The method of claim 1 wherein the subject is a patient with hemophilia A or B.

7. The method of claim 1 wherein the subject is a hemochromatosis patient.

8. The method of claim 1 wherein the compound is administered to the subject intravenously, topically, intradermally, intramuscularly, transdermally, subcutaneously, intranasally, parenterally, intrathecally, vaginally, rectally, colorectally, orally, intracranially, retroorbitally, or intrasternally.

9. The method of claim 1 wherein the method reduces or ameliorates tingling or tightness in the joint, joint pain, difficult joint movement, joint effusion, joint swelling, joint fusion, erosion of joint cartilage, subchondral changes, subchondral cyst formation, synovial hyperplasia, synovial inflammation, joint fibrosis, ankylosis, hemarthrosis, paresthesias, joint destruction, and hemosiderosis in the subject.

10. The method of claim 1 wherein the subject is co-administered with one or more additional therapeutic agents selected from the group consisting of coagulation factor VIIa, coagulation factor VIII, coagulation factor IX, acetaminophen, steroids, hyaluronic acid, glucosamine, chondroitin, shea nut oil extract (shea butter), desmopressin, anti-hemophilic factor recombinant, anti-inhibitor coagulant complex, antifibrinolytic agents, rituximab, chelation therapy, and nonsteroidal anti-inflammatory drugs (NSAIDs) including COX-2 inhibitors.

11. A method for the treatment and/or prevention of hemophilic arthropathy in a subject, comprising administering to the subject in need of such treatment and/or prevention a therapeutically effective amount of a compound selected from the group consisting of diacerein, rhein, monoacetylrhein, and pharmaceutically acceptable salts or esters or prodrugs thereof.

12. The method of claim 11 wherein the compound is administered at a dose of about 5 to 500 mg per day.

13. The method of claim 11 wherein the subject is a hemophilia patient.

14. The method of claim 11 wherein the subject is a patient with hemophilia A or B.

15. The method of claim 11 wherein the compound is administered to the subject intravenously, topically, intradermally, intramuscularly, transdermally, subcutaneously, intranasally, parenterally, intrathecally, vaginally, rectally, colorectally, orally, intracranially, retroorbitally, or intrasternally.

16. The method of claim 11 wherein the method reduces or ameliorates tingling or tightness in the joint, joint pain, difficult joint movement, joint effusion, joint swelling, joint fusion, erosion of joint cartilage, subchondral changes, subchondral cyst formation, synovial hyperplasia, synovial inflammation, joint fibrosis, ankylosis, hemarthrosis, paresthesias, joint destruction, and hemosiderosis in the subject.

17. The method of claim 11 wherein the subject is co-administered with one or more additional therapeutic agents selected from the group consisting of coagulation factor VIIa, coagulation factor VIII, coagulation factor IX, acetaminophen, steroids, hyaluronic acid, glucosamine, chondroitin, shea nut oil extract (shea butter), desmopressin, anti-hemophilic factor recombinant, anti-inhibitor coagulant complex, antifibrinolytic agents, rituximab, chelation therapy, and nonsteroidal anti-inflammatory drugs (NSAIDs) including COX-2 inhibitors.

18. A method for the treatment and/or prevention of hemochromatosis arthropathy in a subject, comprising administering to the subject in need of such treatment and/or prevention a therapeutically effective amount of a compound selected from the group consisting of diacerein, rhein, monoacetylrhein, and pharmaceutically acceptable salts or esters or prodrugs thereof.

19. The method of claim 18 wherein the subject is a hemochromatosis patient.

20. The method of claim 18 wherein the method reduces or ameliorates tingling or tightness in the joint, joint pain, difficult joint movement, joint effusion, joint swelling, joint fusion, erosion of joint cartilage, subchondral changes, subchondral cyst formation, synovial hyperplasia, synovial inflammation, joint fibrosis, ankylosis, hemarthrosis, paresthesias, joint destruction, and hemosiderosis in the subject.

21. The method of claim 18 wherein the subject is co-administered with one or more additional therapeutic agents selected from the group consisting of coagulation factor VIIa, coagulation factor VIII, coagulation factor IX, acetaminophen, steroids, hyaluronic acid, glucosamine, chondroitin, shea nut oil extract (shea butter), desmopressin, anti-hemophilic factor recombinant, anti-inhibitor coagulant complex, antifibrinolytic agents, rituximab, chelation therapy, and nonsteroidal anti-inflammatory drugs (NSAIDs) including COX-2 inhibitors.
